(19) United States
(12) Patent Application Publication
(10) Pub. No.: US 2024/0207358 A1
(43) Pub. Date: Jun. 27, 2024

(54) METHODS OF TREATING SICKLE CELL DISORDER AND RELATED CONDITIONS

(71) Applicant: University of Connecticut, Farmington, CT (US)

(72) Inventors: LIPING XIAO, Farmington, CT (US); Marja HURLEY, Farmington, CT (US); Tannin SCHMIDT, Farmington, CT (US)

(21) Appl. No.: 18/288,244

(22) PCT Filed: May 10, 2022
(86) PCT No.: PCT/US2022/028470

§ 371 Date: Oct. 25, 2023

Related U.S. Application Data
(60) Provisional application No. 63/187,148, filed on May 11, 2021.

(51) Int. Cl. A61K 38/17; A61P 7/06

(52) U.S. Cl. CPC A61K 38/1709; A61P 7/06

(57) ABSTRACT

The present disclosure is directed to administration of native or recombinant human PRG4 (rhPRG4) as a means to treat sickle cell disease or other disorders in subjects in need of treatment thereof.

### RELATED APPLICATION INFORMATION

This application claims priority to U.S. Application No. 63/187,148, filed on May 11, 2021, the contents of which is herein incorporated by reference in its entirety.

### STATEMENT OF GOVERNMENT SUPPORT

This invention was made with government support under grant number HL 147048 awarded by the National Institutes of Health. The government has certain rights in the invention.

### FIELD

The present disclosure relates to methods of treating sickle cell disease by administering to a subject in need of treatment thereof an effective amount of PRG4. In another aspect, the present disclosure relates to methods of treating anemia by administering to a subject in need of treatment thereof an effective amount of PRG4.

### BACKGROUND

Sickle-cell disease (SCD), also known as sickle-cell anemia (SCA) and drepanocytosis, a genetic disorder affecting 1 in 400 African Americans and up to 2% of the population in some areas of Africa, results from the production of an abnormal type of hemoglobin that polymerizes (aggregates) leading to detrimental shape changes in red blood cells (sickling) and significant morbidity and mortality in patients.

Polymerization of sickle hemoglobin (HbS) in the red blood cells of patients with SCD leads to rigid red cells which occlude blood vessels, leading to pain, strokes, organ damage, susceptibility to infection and early death. Present methods known in the art that have been shown to alter the severity of the disorder are complex and labor intensive therapies: 1) bone marrow transplantation; 2) routine blood transfusions; or 3) hydroxyurea, a drug which indirectly (and incompletely) prevents HbS polymerization by inducing the production of another type of hemoglobin (fetal hemoglobin). Accordingly, treatments for SCD are lacking and focus mainly on palliative or symptomatic therapy. Therefore, there remains a need in the art for improved methods for treating sickle cell disorder and the severity of its symptoms.

### SUMMARY

In one aspect, the present disclosure relates to methods treating sickle cell disease, wherein the method comprises administering PRG4 (e.g., full-length PRG4) or a fragment thereof to a subject in need thereof. In some aspects, the subject is suffering from chronic sickle cell disease. In yet other aspects, the subject is suffering from acute sickle cell disease.

In some aspects, the PRG4 administered to a subject suffering from SCD is native human PRG4. In some aspects, the native human PRG4 is full length native PRG4. In some aspects, the PRG4 administered to a subject suffering from SCD is recombinant human PRG4 (rhPRG4). In other aspects, the recombinant human PRG4 is full length recombinant PRG4. In other aspects, the human native or recombinant human PRG4 is a fragment of PRG4. The fragment of PRG4 can have a length of 5 amino acids, 10 amino acids, 15 amino acids, 20 amino acids, 30 amino acids, 35 amino acids, 40 amino acids, 45 amino acids, 50 amino acids, 55 amino acids, 60 amino acids, 65 amino acids, 70 amino acids, 75 amino acids, 100 amino acids, 200 amino acids, 300 amino acids, 400 amino acids, 500 amino acids, 600 amino acids, 700 amino acids, 800 amino acids, 900 amino acids, 1000 amino acids, 1100 amino acids, 1200 amino acids, 1300 amino acids, or 1400 amino acids.

In another aspect, disclosed the present disclosure relates to methods for treating osteoarthropathy, wherein the method comprises administering PRG4 or a fragment thereof to a subject in need thereof, wherein the subject has sickle cell disease. In some embodiments, the osteoarthropathy is selected from infarction of bone and bone marrow, compensatory bone marrow hyperplasia, secondary osteomyelitis, secondary growth defects, intravascular thrombosis, osteonecrosis, degenerative bone and joint destruction, osteolysis, articular disintegration, myelosclerosis, periosteal reaction, Reynold sign or codfish vertebrae, Dystrophic medullary calcification, bone-within-bone appearance, decreased density of the skull, decreased thickness of outer table of skull due to widening of diploe, hair on-end striations of the calvaria, osteoporosis sometimes leading to biconcave vertebrae, coarsening of trabeculae in long and flat bones, pathologic fractures, bone shortening, epiphyseal deformity with cupped metaphysis, peg-in-hold defect of distal femur, and decreased height of vertebrae. In other embodiments, the osteoarthropathy is selected from osteonecrosis, dactylitis, and osteomyelitis.

In some aspects, the PRG4 administered to a subject suffering from osteoarthropathy is native human PRG4. In some aspects, the native human PRG4 is full length native PRG4. In some aspects, the PRG4 administered to a subject suffering from osteoarthropathy is recombinant human PRG4 (rhPRG4). In other aspects, the recombinant human PRG4 is full length recombinant PRG4. In other aspects, the human native or recombinant human PRG4 is a fragment of the native or recombinant human PRG4. The fragment of PRG4 can have a length of 5 amino acids, 10 amino acids, 15 amino acids, 20 amino acids, 30 amino acids, 35 amino acids, 40 amino acids, 45 amino acids, 50 amino acids, 55 amino acids, 60 amino acids, 65 amino acids, 70 amino acids, 75 amino acids, 100 amino acids, 200 amino acids, 300 amino acids, 400 amino acids, 500 amino acids, 600 amino acids, 700 amino acids, 800 amino acids, 900 amino acids, 1000 amino acids, 1100 amino acids, 1200 amino acids, 1300 amino acids, or 1400 amino acids.

In yet a further aspect, the present disclosure relates to methods of treating anemia, wherein the method comprises administering PRG4 or a fragment thereof to a subject in need thereof. In some aspects, the subject is suffering from anemia caused by sickle cell disease. In yet other aspects, the subject is suffering from anemia that is not caused by sickle cell disease. For example, the anemia can be iron refractory iron deficiency anemia.

In some aspects, the PRG4 administered to a subject suffering from anemia is native human PRG4. In some aspects, the native human PRG4 is full length native PRG4. In some aspects, the PRG4 administered to a subject suffering from anemia is recombinant human PRG4 (rhPRG4). In other aspects, the recombinant human PRG4 is full length recombinant PRG4. In other aspects, the human native or recombinant human PRG4 is a fragment of native or recombinant human PRG4. The fragment of PRG4 can have a length of 5 amino acids, 10 amino acids, 15 amino acids, 20 amino acids, 30 amino acids, 35 amino acids, 40 amino acids, 45 amino acids, 50 amino acids, 55 amino acids, 60 amino acids, 65 amino acids, 70 amino acids, 75 amino acids, 100 amino acids, 200 amino acids, 300 amino acids, 400 amino acids, 500 amino acids, 600 amino acids, 700 amino acids, 800 amino acids, 900 amino acids, 1000 amino acids, 1100 amino acids, 1200 amino acids, 1300 amino acids, or 1400 amino acids.

In other embodiments, in the methods described herein, PRG4 is administered to the subject as a pharmaceutically acceptable composition. In some embodiments, the pharmaceutically acceptable composition comprises a pharmaceutically acceptable carrier or diluent.

### BRIEF DESCRIPTION OF THE FIGURES

FIG. 1 shows recombinant PRG4 (rhPRG4) treatment ameliorates decreased Hb and OA phenotype in SCD mice. FIG. 1A is a bar graph showing the Hb level in peripheral blood. n=6-10 mice/group. *: p<0.05; two way ANOVA. Data are mean±SE. FIG. 1B is Safranin O staining of a knee joint.

FIG. 2 shows flow cytometric analysis of HbF synthesis in circulating RBCs from vehicle or PRG4 treated Ctrl and SCD mice. Flow cytometry analysis shows rhPRG4 treatment increased the percentage of HbF-high RBCs in SCD mice. Data are mean±SE. n=3-5 mice/group. *: p<0.05; two way ANOVA. High levels of fetal hemoglobin (HbF) protect from complications of sickle cell disease and lead to improved survival as described in Example 2.

FIG. 3 shows rhPRG4 treatment increases serum and knee joint PRG4 in SCD mice as described in Example 3. FIG. 3A is a bar graph showing serum PRG4 level by AlphaLISA analysis. n=5 mice/group. *: p<0.05; t test and correct for multiple comparison. FIG. 3B is immunohistochemistry staining of PRG4 in knee joint.

FIG. 4 shows enlarged bone marrow area in SCD mice was partially rescued after rhPRG4 treatment. FIG. 4A is representative uCT image of each group. FIG. 4B is bar graph showing increased marrow area (Ma.Ar) and endocortical perimeter (Ec.Pm) are rescued with rhPRG4 treatment.

FIGS. 5A-5C show flow cytometric analysis of BM erythroid lineage cell apoptosis in Vehicle or PRG4 treated Ctrl and SCD mice. FIG. 5A is a bar graph showing the total apoptotic erythroid lineage cells. Percentage of Apotracker+ in Ter119+ cells were counted. FIG. 5B is a bar graph showing the early apoptotic erythroid lineage cells. Apotracker+/PI− in Ter119+ cells were counted. FIG. 5C is the late apoptotic erythroid lineage cells. Apotracker+/PI+ in Ter119+ cells were counted.

FIG. 6 shows decreased trabecular thickness in SCD was rescued after rhPRG4 treatment.

### DETAILED DESCRIPTION

Hemolysis is a fundamental feature of sickle cell disease (SCD) that induces inflammation and vasculopathy that contributes to its pathophysiology and multiple clinical complications including osteoarthritis (OA). Degenerative osteoarthropathy is a form of OA prevalent in individuals with SCD and is associated with significant morbidity, with no effective therapy. Inflammation is implicated in the pathogenesis of most complications seen in both SCD patients and OA. Proteoglycan 4 (PRG4) is necessary for maintaining articular cartilage integrity and an antagonist to toll like receptors 2 and 4.

In one aspect, the present disclosure is directed to administration of PRG4 (native or recombinant) to a subject in need of treatment thereof as a means to ameliorate, sickle cell disease and/or anemia and/or mitigate osteoarthropathy. Accordingly, disclosed herein are methods of treating sickle cell disease by administering PRG4 (e.g., native or rhPRG4) to a subject in need thereof. Also disclosed herein are methods for treating osteoarthropathy by administering PRG4 (e.g., native or rhPRG4) to a subject in need thereof, wherein the subject has sickle cell disease. Also disclosed herein are methods for treating inflammation by administering PRG4 (e.g., native or rhPRG4) to a subject in need thereof, wherein the subject has sickle cell disease. In still further aspects, disclosed herein are methods for treating anemia by administering PRG4 (e.g., native or rhPRG4) to a subject in need thereof. In some aspects, the anemia may be caused by sickle cell disease. In other aspects, the anemia may not be caused by sickle cell disease.

In the aspects disclosed above, the PRG4 administered in the above methods can be native or recombinant PRG4. In some aspects, the PRG4 is native human PRG4. In other aspects, the PRG4 is a recombinant human PRG4 (e.g., rhPRG4). In some aspects, the native or recombinant human PRG4 is full length native or recombinant human PRG4. In other aspects, the native or recombinant human PRG4 comprises a fragment of the full-length native or recombinant human PRG4. In some aspects, the PRG4 fragment has a length of 5 amino acids, 10 amino acids, 15 amino acids, 20 amino acids, 30 amino acids, 35 amino acids, 40 amino acids, 45 amino acids, 50 amino acids, 55 amino acids, 60 amino acids, 65 amino acids, 70 amino acids, 75 amino acids, 100 amino acids, 200 amino acids, 300 amino acids, 400 amino acids, 500 amino acids, 600 amino acids, 700 amino acids, 800 amino acids, 900 amino acids, 1000 amino acids, 1100 amino acids, 1200 amino acids, 1300 amino acids, or 1400 amino acids.

### Definitions

Throughout the present specification and the accompanying claims the words “comprise,” “include,” and “have” and variations thereof such as “comprises,” “comprising,” “includes,” “including,” “has,” and “having” are to be interpreted inclusively. That is, these words are intended to convey the possible inclusion of other elements or integers not specifically recited, where the context allows. No language in the specification should be construed as indicating any non-claimed element essential to the practice of the disclosure

The terms “a,” “an,” and “the” and similar referents used in the context of describing the disclosure (especially in the context of the following claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, each individual value is incorporated into the specification as if it were individually recited herein. Ranges may be expressed herein as from “about” (or “approximately”) one particular value, and/or to “about” (or “approximately”) another particular value. When such a range is expressed, another embodiment includes from the one particular value and/or to the other particular value. Similarly, when values are expressed as approximations, by use of the antecedent “about” or “approximately” it will be understood that the particular value forms another embodiment. It will be further understood that the endpoints of each of the ranges are disclosed both in relation to the other endpoint, and independently of the other endpoint.

All methods described herein can be performed in any suitable order unless otherwise indicated herein or otherwise clearly contradicted by context. Further, all methods described herein and having more than one step can be performed by more than one person or entity. Thus, a person or an entity can perform step (a) of a method, another person or another entity can perform step (b) of the method, and a yet another person or a yet another entity can perform step (c) of the method, etc. The use of any and all examples, or exemplary language (e.g., “such as”) provided herein is intended merely to better illuminate the disclosure and does not pose a limitation on the scope of the disclosure otherwise claimed.

Units, prefixes, and symbols are denoted in their Systeme International de Unites (SI) accepted form.

Groupings of alternative elements or embodiments of the disclosure disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

The headings used herein are for organizational purposes only and are not meant to be used to limit the scope of the description or the claims, which can be had by reference to the specification as a whole. Accordingly, the terms defined immediately below are more fully defined by reference to the specification in its entirety.

Illustrations are for the purpose of describing a preferred embodiment of the disclosure and are not intended to limit the disclosure thereto.

As used herein, the term “about” refers to a range of values of plus or minus 10% of a specified value. For example, the phrase “about 200” includes plus or minus 10% of 200, or from 180 to 220, unless clearly contradicted by context.

As used herein, the term “administering” means the actual physical introduction of a composition into or onto (as appropriate) a host or cell. Any and all methods of introducing the composition into the host or cell are contemplated according to the disclosure; the method is not dependent on any particular means of introduction and is not to be so construed. Means of introduction are well-known to those skilled in the art, and also are exemplified herein.

The terms “modulate,” “modulation,” or “modulating” are art-recognized and refer to up-regulation (i.e., activation, stimulation, increase), or down-regulation (i.e., inhibition, suppression, reduction, or decrease) of a response, or the two in combination or apart.

As used herein, “optional” or “optionally” means that the subsequently described event or circumstance may or may not occur, and that the description includes instances where said event or circumstance occurs and instances where it does not.

As used herein, the term “pharmaceutically acceptable” refers to compositions that are physiologically tolerable and do not typically produce an allergic or similar untoward reaction when administered to a subject, preferably a human subject. Preferably, as used herein, the term “pharmaceutically acceptable” means approved by a regulatory agency of a federal or state government or listed in the U.S. Pharmacopeia or other generally recognized pharmacopeia for use in animals, and more particularly in humans.

As used herein, the term “subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc.) and a human). In some embodiments, the subject may be a human or a non-human. In some embodiments, the subject is a human. The subject or patient may be undergoing other forms of treatment. In some embodiments, the subject is a human that may be undergoing other forms of treatment.

As used herein, the term “substantially decreased” and grammatical equivalents thereof refer to a level, amount, concentration of a parameter, such as a chemical compound, a metabolite, a nucleic acid, a polypeptide or a physical parameter (pH, temperature, viscosity, etc.) measured in a sample that has a decrease of at least 10%, preferably about 20%, more preferable about 40%, even more preferable about 50% and still more preferably a decrease of more than 75% when compared to the level, amount, or concentration of the same chemical compound, nucleic acid, polypeptide or physical parameter in a control sample.

As used herein, the term “substantially increased” and grammatical equivalents thereof refer to a level, amount, concentration of a parameter, such as a chemical compound, a metabolite, a nucleic acid, a polypeptide or a physical parameter (pH, temperature, viscosity, etc.) measured in a sample that has an increase of at least 30%, preferably about 50%, more preferable about 75%, and still more preferably an increase of more than 100% when compared to the level, amount, or concentration of the same chemical compound, nucleic acid, polypeptide, or physical parameter in a control sample.

As used herein, the terms “treat,” “treating,” and “treatment” include inhibiting the pathological condition, disorder, or disease, e.g., arresting or reducing the development of the pathological condition, disorder, or disease or its clinical symptoms; or relieving the pathological condition, disorder, or disease, e.g., causing regression of the pathological condition, disorder, or disease or its clinical symptoms. These terms also encompass therapy and cure. Treatment means any way the symptoms of a pathological condition, disorder, or disease are ameliorated or otherwise beneficially altered. Preferably, the subject in need of such treatment is a mammal, preferably a human.

### PRG4 Protein

PRG4, also referred to as lubricin, is a lubricating polypeptide, which in humans is expressed from the megakaryocyte stimulating factor (MSF) gene, also known as PRG4 (see NCBI Accession Number AK131434-U70136). Lubricin is a ubiquitous, endogenous glycoprotein that coats the articulating surfaces of the body. Lubricin is highly surface active molecule (e.g., holds onto water), that acts primarily as a potent cytoprotective, anti-adhesive and boundary lubricant. The molecule has a long, central mucin-like domain located between terminal protein domains that allow the molecule to adhere and protect tissue surfaces. Its natural form, in all mammals investigated, contains multiple repeats of an amino acid sequence which is at least 50% identical to KEPAPTT. Natural lubricin typically comprises multiple redundant forms of this repeat, which typically includes proline and threonine residues, with at least one threonine being glycosylated in most repeats. The threonine anchored O-linked sugar side chains are critical for lubricin's boundary lubricating function. The side chain moiety typically is a β(1-3)Gal-GalNAc moiety, with the β(1-3)Gal-GalNAc typically capped with sialic acid or N-acetylneuraminic acid. The polypeptide also contains N-linked oligosaccharides. The gene encoding naturally occurring full length lubricin contains 12 exons, and the naturally-occurring MSF gene product contains 1,404 amino acids (including the secretion sequence) with multiple polypeptide sequence homologies to vitronectin including hemopexin-like and somatomedin-like regions. Centrally located exon 6 contains 940 residues. Exon 6 encodes the repeat rich, O-glycosylated mucin-like domain.

The amino acid sequence of the protein backbone of lubricin may differ depending on alternative splicing of exons of the human MSF gene. This robustness against heterogeneity was exemplified when researchers created a recombinant form of lubricin missing 474 amino acids from the central mucin domain, yet still achieved reasonable, although muted, lubrication (Flannery et al., Arthritis Rheum 2009; 60(3):840-7). PRG4 has been shown to exist not only as a monomer but also as a dimer and multimer disulfide-bonded through the conserved cysteine-rich domains at both N- and C-termini. Lubris, LLC has developed a full-length recombinant form of human lubricin. The molecule is expressed using the Selexis Chinese hamster ovary cell line (CHO-M), with a final apparent molecular weight of 450-600 kDa, with polydisperse multimers frequently measuring at 1,000 kDa or more, all as estimated by comparison to molecular weight standards on SDS tris-acetate 3-8% polyacrylamide gels. Of the total glycosylations, about half comprise two sugar units (GalNAc-Gal), and half three sugar units (GalNAc-Gal-Sialic acid). This method of recombinant human PRG4 production is disclosed in International Patent Publication No. WO 2015/061488.

Any one or more of various native and recombinant PRG4 proteins and isoforms may be utilized in the various embodiments described herein. For instance, U.S. Pat. Nos. 6,433,142; 6,743,774; 6,960,562; 7,030,223, and 7,361,738 disclose how to make various forms of human PRG4 expression product. In some aspects, the PRG4 used in the methods described herein is full length, glycosylated, human recombinant PRG4, or lubricin, expressed from CHO cells. This protein comprises 1,404 amino acids including a central exon comprising repeats of the sequence KEPAPTT variously glycosylated with O-linked 0 (1-3) Gal-GalNAc oligosaccharides, and including N and C-terminal sequences with homology to vitronectin. The molecule is polydisperse with the glycosylation pattern of individual molecules varying, and can comprise monomeric, dimeric, and multimeric species. In other aspects, the PRG4 used in the methods described herein is full length, glycosylated, human native PRG4. In still other aspects, the native or recombinant human PRG4 comprises a fragment of the full length human native or recombinant PRG4. In some aspects, the PRG4 fragment has a length of 5 amino acids, 10 amino acids, 15 amino acids, 20 amino acids, 30 amino acids, 35 amino acids, 40 amino acids, 45 amino acids, 50 amino acids, 55 amino acids, 60 amino acids, 65 amino acids, 70 amino acids, 75 amino acids, 100 amino acids, 200 amino acids, 300 amino acids, 400 amino acids, 500 amino acids, 600 amino acids, 700 amino acids, 800 amino acids, 900 amino acids, 1000 amino acids, 1100 amino acids, 1200 amino acids, 1300 amino acids, or 1400 amino acids.

As used herein, the term “PRG4” is used interchangeably with the term “lubricin.” Broadly, these terms refer to any functional isolated or purified native or recombinant PRG4 proteins, homologs, functional fragments, isoforms, and/or mutants thereof. All useful molecules comprise the sequence encoded by exon 6, or homologs or truncated versions thereof, for example, versions with fewer repeats within this central mucin-like KEPAPIT-repeat domain, preferably together with O-linked glycosylation. All useful molecules also comprise at least the biological active portions of the sequences encoded by exons 1-5 and 7-12, i.e., sequences responsible for imparting to the molecule its affinity for ECM and endothelial surfaces. In certain embodiments, a preferred PRG4 protein has an average molar mass of between 50 kDa and 500 kDa, preferably between 224 to 467 kDa, comprising one or more biological active portions of the PRG4 protein, or functional fragments, such as a lubricating fragment, or a homolog thereof. In a more preferred embodiment, a PRG4 protein comprises monomers of average molar mass of between 220 kDa to about 280 kDa.

Methods for isolation, purification, and recombinant expression of a proteins such as PRG4 protein are well known in the art. In certain cases, the method starts with cloning and isolating mRNA and cDNA encoding PRG4 proteins or isoforms using standard molecular biology techniques, such as PCR or RT-PCR. The isolated cDNA encoding the PRG4 protein or isoform is then cloned into an expression vector and expressed in a host cell for producing recombinant PRG4 protein, and isolated from the cell culture supernatant. A method for production of recombinant human PRG4 is provided in International Patent Publication No. WO 2015/061477.

PRG4 is naturally present in synovial fluid and on the surface (superficial layer) of articular cartilage and therefore plays an important role in joint lubrication and synovial homeostasis. The function of PRG4 heretofore has been almost entirely associated with reduction of friction and prevention of wear between articulating joints and lubrication of interfacing tissues such as between the surface of the eye and eyelid. In the present disclosure, it has been discovered that treatment with PRG4 can substantially increase peripheral blood hemoglobin (Hb). Accordingly, the methods disclosed herein typically include administering PRG4 (e.g., rhPRG4 or native PRG4), or a pharmaceutically acceptable composition thereof, to a subject with sickle cell disease to increase expression of Hb in the subject.

Additionally, it has also been discovered that treatment of a subject with sickle cell disease with PRG4 (e.g., rhPRG4 or native PRG4) improves bone loss, rescues loss of proteoglycan, and rescues thinning of articular cartilage. Accordingly, the methods disclosed herein typically include administering PRG4 (e.g., rhPRG4 or native PRG4), or a pharmaceutically acceptable composition thereof, to a subject with sickle cell disease to improve bone loss, rescue loss of proteoglycan in articular cartilage, and/or rescue thinning of articular cartilage.

### Diseases to be Treated

The disclosed compositions can be used to treat subjects with one or more mutations in the beta-globin gene (HBB gene). Mutations in the beta globin gene can cause sickle cell disease, beta thalassemia, or related diseases or conditions thereof. As discussed in more detail below, mutations in the beta-globin gene can be identified before or after manifestations of a disease's clinical symptoms. The compositions can be administered to a subject with one or more mutations in the beta-globin gene before or after the onset of clinical symptoms. Therefore, in some embodiments, the compositions are administered to a subject that has been diagnosed with one or more mutations in the beta-globin gene, but does not yet exhibit clinical symptoms. In some embodiments, the compositions are administered to a subject that is exhibiting one or more symptoms of a disease, condition, or syndrome associated with, or caused by one or more mutations in the beta-globin gene.

In other aspects, the compositions can be used to treat subjects suffering from anemia. In some aspects, the anemia can be related to SCD. In other aspects, the anemia is caused by a condition or disease other than SCD.

### Sickle Cell Disease

Sickle cell disease (SCD) typically arises from a mutation substituting thymine for adenine in the sixth codon of the beta-chain gene of hemoglobin (i.e., GAG to GTG of the HBB gene). This mutation causes glutamate to valine substitution in position 6 of the Hb beta chain. The resulting Hb, referred to as HbS, has the physical properties of forming polymers under deoxy conditions. SCD is typically an autosomal recessive disorder. Therefore, in some embodiments, the disclosed compositions and methods are used to treat a subject homozygous for an autosomal recessive mutation in beta-chain gene of hemoglobin (i.e., homozygous for sickle cell hemoglobin (HbS)). Also referred to as HbSS disease or sickle cell anemia (the most common form), subjects homozygote for the S globin typically exhibit a severe or moderately sever phenotype and have the shortest survival of the hemaglobinopathies.

Sickle cell trait or the carrier state is the heterozygous form characterized by the presence of around 40% HbS, absence of anemia, inability to concentrate urine (isosthenuria), and hematuria. Under conditions leading to hypoxia, it may become a pathologic risk factor. Accordingly, in some embodiments, the disclosed compositions and methods are used to treat a subject heterozygous for an autosomal recessive mutation in the beta-chain gene of hemoglobin (i.e., heterozygous for HbS).

In some aspects, the subject suffering from SCD is suffering from acute SCD. Subjects with acute SCD typically suffer acute pain, acute chest syndrome, infection, priapism, stroke (including overt thrombotic stroke), cerebral vasculopathy, splenic sequestration or any combinations thereof. In some aspects, acute complications of SCD may occur suddenly and resolve quickly, however, the underlying damage associated with such acute complications may occur and worsen over time.

In yet other aspects, the subject suffering from SCD is suffering long-term or chronic SCD. Subject s with long-term or chronic SCD typically suffer pulmonary hypertension, cardiovascular complications, cerebral vasculopathy, cholelithiasis, renal dysfunction, avascular necrosis of femoral head, recurrent, chronic leg ulcers, silent stroke (e.g., asymptomatic cerebral infarction), depression and anxiety, end organ disease or any combinations thereof.

### Beta-Thalassemia

Beta-thalassemias (β-thalassemias) are a group of inherited blood disorders caused by a variety of mutational mechanisms that result in a reduction or absence of synthesis of β-globin and leading to accumulation of aggregates of unpaired, insoluble α-chains that cause ineffective erythropoiesis, accelerated red cell destruction, and severe anemia. Subjects with beta-thalassemia exhibit variable phenotypes ranging from severe anemia to clinically asymptomatic individuals. The genetic mutations present in β thalassemias are diverse, and can be caused by a number of different mutations. The mutations can involve a single base substitution or deletions or inserts within, near or upstream of the β globin gene. For example, mutations occur in the promoter regions preceding the beta-globin genes or cause production of abnormal splice variants.

Examples of thalassemias include thalassemia minor, thalassemia intermedia, and thalassemia major.

Thalassemia minor refers to thalassemia where only one of beta-globin alleles bears a mutation. Individuals typically suffer from microcytic anemia. Detection usually involves lower than normal MCV value (<80 fL) plus an increase in fraction of Hemoglobin A2 (>3.5%) and a decrease in fraction of Hemoglobin A (<97.5%). Genotypes can be β+/β or β−0/β.

Thalassemia intermedia refers to a thalassemia intermediate between the major and minor forms. Affected individuals can often manage a normal life but may need occasional transfusions, e.g., at times of illness or pregnancy, depending on the severity of their anemia. Genotypes can be β+/β+ or β−0/β.

Thalassemia major refers to a thalassemia where both beta-globin alleles have thalassemia mutations. This is a severe microcytic, hypochromic anemia. If left untreated, it causes anemia, splenomegaly, and severe bone deformities and typically leads to death before age 20. Treatment consists of periodic blood transfusion-caused iron overload. Cure is possible by bone marrow transplantation. Cooley's anemia is named after Thomas Benton Cooley. Genotypes include β+/β−0 or β−0/β−0 or β+/β+.

### Sickle Cell Related Disorders

Although carriers of sickle cell trait do not suffer from SCD, individuals with one copy of HbS and one copy of a gene that codes for another abnormal variant of hemoglobin, such as HbC or Hb beta-thalassemia, have a less severe form of the disease. For example, another specific defect in beta-globin causes another structural variant, hemoglobin C (HbC). Hemoglobin C (abbreviated as Hb C or HbC) is an abnormal hemoglobin in which substitution of a glutamic acid residue with a lysine residue at the 6^{th} position of the β-globin chain has occurred. A subject that is a double heterozygote for HbS and HbC (HbSC disease) is typically characterized by symptoms of moderate clinical severity.

Another common structural variant of beta-globin is hemoglobin E or hemoglobin E (HbE). HbE is an abnormal hemoglobin in which substitution of a glutamic acid residue with a lysine residue at the 26^{th} position of the β-globin chain has occurred. A subject that is a double heterozygote for HbS and HbE has HbS/HbE syndrome, which usually causes a phenotype similar to HbS/b+ thalassemia, discussed below.

Some mutations in the beta-globin gene can cause other structural variations of hemoglobin or can cause a deficiency in the amount of β-globin being produced. These types of mutations are referred to as beta-thalassemia mutations.

The absence of beta-globin is referred to as beta-zero (β−0) thalassemia. A subject that is a double heterozygote for HbS and β−0 thalassemia (i.e., HbS/β−0 thalassemia) can suffer symptoms clinically indistinguishable from sickle cell anemia.

A reduced amount of beta-globin is referred to as β-plus (β+) thalassemia. A subject that is a double heterozygote for HbS and β+ thalassemia (i.e., HbS/β+ thalassemia) can have mild-to-moderate severity of clinical symptoms with variability among different ethnicities.

Rare combinations of HbS with other abnormal hemoglobins include HbD Los Angeles, G-Philadelphia, HbO Arab, and others.

Therefore, in some embodiments, the disclosed compositions and methods are used to treating a subject with an HbS/β−0 genotype, an HbS/β+ genotype, an HBSC genotype, an HbS/HbE genotype, an HbD Los Angeles genotype, a G-Philadelphia genotype, or an abHbO Arab genotype.

### Symptoms of Sickle Cell Disease, Beta-thalassemias, and Related and Other Disorders

In some embodiments, the compositions disclosed, herein are administered to a subject in an effective amount to treatment one or more symptoms of sickle cell disease, a beta-thalassemia, or a related disorder, such as, for example, anemia, inflammation, and osteoarthropathy.

### Anemia

In some aspects, the present disclosure relates to administered the disclosed composition to a subject in an effective amount to treat anemia. Anemia results when a subject does not have enough red blood cells or the subject's red blood cells do not function properly. In some aspects, the anemia is related to or the result of SCD. Specifically, SCD is a form of hemolytic anemia, with red cell survival of around 1-20 days. Approximately one third of the hemolysis occurs intravascularly, releasing free hemoglobin (plasma free hemoglobin [PFH]) and arginase into plasma. PFH has been associated with endothelial injury including scavenging nitric oxide (NO), proinflammatory stress, and coagulopathy, resulting in vasomotor instability and proliferative vasculopathy. A hallmark of this proliferative vasculopathy is the development of pulmonary hypertension in adulthood.

In other aspects, the anemia is not related to or the result of SCD. In some aspects, the etiology of the anemia may be unknown. In other aspects, the anemia may be the result of or caused by a diet lacking in vitamins and minerals, medical treatment (e.g., chemotherapy, other medical treatment (e.g., small molecules, large molecules (e.g., biologics) or a combination of small and large molecules), pregnancy, menstruation, intestinal disorders, internal bleeding, sudden blood loss, or any combinations thereof. In some aspects, the subject suffering from the anemia may be non-responsive or unresponsive to treatment with iron supplements, such as, for example, a suffering from iron refractory iron deficiency anemia. Iron-refractory iron deficiency anemia is a type of iron deficiency anemia that does not improve with oral iron treatment.

### Inflammation

Sickle red blood cells (RBCs) can exacerbate inflammation and thus cause pain in subjects with SCD. Moreover, the inflammatory response in subjects with SCD makes them more susceptible to invasive infections, have pain, and are at a significantly higher risk for acute stroke or chronic cerebral ischemia.

### Osteoarthropathy

Skeletal manifestations include, but are not limited to, infarction of bone and bone marrow, compensatory bone marrow hyperplasia, secondary osteomyelitis, secondary growth defects, intravascular thrombosis, osteonecrosis (avascular necrosis/aseptic necrosis), degenerative bone and joint destruction, osteolysis (in acute infarction), Articular disintegration, myelosclerosis, periosteal reaction (unusual in the adult), H vertebrae (steplike endplate depression also known as the Reynold sign or codfish vertebrae), Dystrophic medullary calcification, bone-within-bone appearance, decreased density of the skull, decreased thickness of outer table of skull due to widening of diploe, hair on-end striations of the calvaria, osteoporosis sometimes leading to biconcave vertebrae, coarsening of trabeculae in long and flat bones, and pathologic fractures, bone shortening (premature epiphyseal fusion), epiphyseal deformity with cupped metaphysis, peg-in-hold defect of distal femur, and decreased height of vertebrae (short stature and kyphoscoliosis).

In particular, is a frequent complication in sickle cell disease, with a painful and debilitating pattern. It is generally insidious and progressive, affecting mainly the hips (femur head) and shoulders (humeral head). Dactylitis, also known as hand-foot syndrome, is an acute vaso-occlusive complication characterized by pain and edema in both hands and feet, frequently with increased local temperature and erythema. Osteomyelitis is the most common form of joint infection in sickle cell disease.

### Administration of PRG4

While PRG4 is produced naturally within the body, the effects of the disclosure are observed when exogenous PRG4 is administered to a subject. Accordingly, in one embodiment, the PRG4 administered to a subject is exogenous or native human PRG4. In other embodiments, the PRG4 administered to the subject is recombinant human PRG4 (rhPRG4). In other aspects, the PRG4 administered to a subject is a full-length native or recombinant human PRG4. In still other aspects, the native or recombinant human PRG4 comprises a fragment of the full-length human native or recombinant PRG4. In some aspects, the PRG4 fragment has a length of 5 amino acids, 10 amino acids, 15 amino acids, 20 amino acids, 30 amino acids, 35 amino acids, 40 amino acids, 45 amino acids, 50 amino acids, 55 amino acids, 60 amino acids, 65 amino acids, 70 amino acids, 75 amino acids, 100 amino acids, 200 amino acids, 300 amino acids, 400 amino acids, 500 amino acids, 600 amino acids, 700 amino acids, 800 amino acids, 900 amino acids, 1000 amino acids, 1100 amino acids, 1200 amino acids, 1300 amino acids, or 1400 amino acids.

The amount of PRG4 administered will depend on variables such as the severity of symptoms a subject with sickle cell disease is experiencing. For example, the amount of PRG4 administered in a subject with sickle cell disease having osteoarthropathy may be based on the level of bone or joint pain experienced by the subject. Additional factors that are considered in determining the amount of PRG4 to administer to a given subject are the seriousness of the sickle cell disease (e.g., the amount of pain the subject is experiencing), the overall health of the patient, the pharmaceutical formulation, and the route of administration. The initial dosage can be increased beyond the upper level in order to rapidly achieve the desired blood-level or tissue level. Alternatively, the initial dosage can be smaller than the optimum, and the dosage may be progressively increased during the course of treatment. Alternatively, the initial dosage can be smaller than the optimum, and the dosage may be progressively increased during the course of treatment. The optimal dose can be determined by routine experimentation.

In some embodiments, the PRG4 is administered to a subject in an amount sufficient to treat the SCD, anemia, inflammation, osteoarthropathy, or any combinations thereof. In some aspects, the PRG4 is administered to a subject in an amount sufficient to treat the SCD (e.g., whether acute or chronic). In some aspects, the PRG4 is administered to a subject in an amount sufficient to treat the anemia (e.g., whether the anemia is the result of SCD or not the result of SCD). In some aspects, the PRG4 is administer to a subject in an amount sufficient to treat iron-refractory iron deficiency anemia. In still further aspects, the PRG4 is administered to a subject in an amount sufficient to treat inflammation. In still another aspect, the PRG4 is administered to a subject in an amount to treat osteoarthropathy.

In some further embodiments, the PRG4 is administered in an amount that is insufficient to provide boundary lubrication, but sufficient to treat joint pain or allodynia. In one embodiment, the PRG4 is administered in an amount that is insufficient to provide boundary lubrication, but sufficient to reduce inflammation associated with sickle cell disease.

Accordingly, in some embodiments, a therapeutically effective amount of PRG4 for administration according to the methods and conditions described herein in the range of about 0.1 μg/kg to about 4000 μg/kg, or about 0.1 μg/kg to about 1000 μg/kg, or about 0.1 μg/kg to about 100 μg/kg, or about 0.1 μg/kg to about 50 μg/kg. In some embodiments, the therapeutically effective amount of PRG4 administered is in the range of about 0.1 mg/kg to about 100 mg/kg, or about 1 mg/kg to about 100 mg/kg, or about 1 mg/kg to about 10 mg/kg.

In further embodiments, PRG4 is administered systemically in an amount sufficient to achieve a concentration of PRG4 in a subject of at least about 50 μg/mL, at least about 100 μg/mL, at least about 150 μg/mL, at least about 200 μg/mL, at least about 250 μg/mL, at least about 300 μg/mL, at least about 350 μg/mL, at least about 400 μg/mL, at least about 450 μg/ml, at least about 500 μg/ml, at least about 550 μg/ml, at least about 600 μg/ml, at least about 650 μg/ml, at least about 750 μg/ml, at least about 800 μg/ml, at least about 850 μg/ml, at least about 900 μg/ml, at least about 950 μg/ml, or at least about 1000 μg/ml. In certain embodiments, a total amount of 2 mg to 10 mg of PRG4 is administered per dose, e.g., about 2 mg to about 10 mg, about 2 mg to about 5 mg, about 2 mg to about 3 mg, about 3 mg to about 4 mg, about 4 mg to about 5 mg, about 5 mg to about 6 mg, about 6 mg to about 7 mg, about 7 mg to about 8 mg, about 8 mg to about 9 mg, about 9 mg to about 10 mg, or about 5 mg to about 10 mg. In certain embodiments, more than about 10 mg of lubricin is administered per dose. The PRG4 may also be administered intravenously to achieve the desired concentration of PRG4. It is contemplated in this disclosure that the dose of PRG4 used for intravenous administration is at least about 1.5-fold, or at least about 2-fold, or at least about 3-fold, or at least about 4-fold, or at least about 5-fold, or at least about 10-fold higher than dose used for intra-articular administration.

In some aspects, PRG4 may be administered to the patient suffering from sickle cell disease, anemia, inflammation, osteoarthropathy, or any combinations thereof, systemically. In other aspects, PRG4 may be administered to the patient suffering from sickle cell disease, anemia, inflammation, osteoarthropathy, or any combinations thereof, directly or locally (e.g., such as by injection).

Systemic administration of PRG4 is also contemplated by some embodiments of the disclosure. For example, PRG4 may be systemically administered in an enteral manner, such as oral, rectal, sublingual, sublabial, or buccal delivery. PRG4 may be systemically administered in a parenteral manner, such as nasal, by inhalation, intravenous, intramuscular, subcutaneous, intradermal, or transmucosal delivery. A preferred route of systemic administration of PRG4 contemplated herein is intravenous administration. The optimal dose can be determined by routine experimentation depending on variables such as the level of pain the patient is experiencing, the overall health of the patient, and the pharmaceutical formulation.

For administration, a dose between about 0.1 mg/kg and about 100 mg/kg, alternatively between about 0.5 mg/kg and about 50 mg/kg, alternatively, between about 1 mg/kg and about 25 mg/kg, alternatively between about 2 mg/kg and about 15 mg/kg, alternatively between about 5 mg/kg and about 15 mg/kg, alternatively between about 0.05 mg/kg and about 1.50 mg/kg can be administered to a subject and may be given, for example, once daily, once weekly, twice weekly, three times weekly, once every other week, once every third week, or once monthly per treatment cycle.

In some aspects, when the PRG4 is being administered to a subject to treat anemia, the PRG4 can be administered in combination with iron supplementation. For example, the subject can be treated with both PRG4 and iron supplements. The order of administration is not critical.

For therapeutic use, the PRG4 administered is preferably combined with a pharmaceutically acceptable carrier. As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration. Suitable carriers include phosphate buffered saline at concentrations ranging from about 1 μg/mL to about 1000 μg/mL, and more preferably 100 μg/mL to 500 μg/mL. Suitable carriers may also include physiological saline, bacteriostatic water, Cremophor EL™ (BASF, Parsippany, N.J.) or phosphate buffered saline (PBS), optionally in admixture with surfactants such as polysorbates. Suitable carriers may also include a solvent or dispersion medium containing, for example, water, ethanol, polyol (for example, glycerol, propylene glycol, and liquid polyethylene glycol), and suitable mixtures thereof. The carrier should be stable under the conditions of manufacture and storage and should be preserved against microorganisms. The use of carriers for pharmaceutically active substances is known in the art. For example, see Remington's Pharmaceutical Sciences, 18th ed. (Mack Publishing Company, 1990).

Useful formulations can be prepared by methods well known in the pharmaceutical arts. For example, see Remington's Pharmaceutical Sciences, 18th ed. (Mack Publishing Company, 1990). Formulation components suitable for parental administration include a sterile diluent such as water for injection, saline solution, fixed oils, polyethylene glycol, glycerin, propylene glycol or other synthetic solvents; antibacterial agents such as benzyl alcohol or methyl paraben; antioxidants such as ascorbic acid or sodium bisulfite; chelating agents such as EDTA; buffers such as acetates, citrates or phosphates; and agents for the adjustment of tonicity such as sodium chloride or dextrose. Lubricin for administration can be present in a dosage unit form and can be prepared by any suitable method and should be formulated to be compatible with its intended route of administration.

In some aspects, PRG4 for administration should be formulated to be compatible with its intended route of administration, for example, intra-articular (IA), intravenous (IV), intramuscular, subcutaneous, intradermal, intranasal, transdermal, topical, transmucosal, oral and rectal administration. The formulation of PRG4 can be presented in a dosage unit form and prepared by any suitable method known in the art. For example, formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl paraben; antioxidants such as ascorbic acid or sodium bisulfite; chelating agents such as EDTA; buffers such as acetates, citrates or phosphates; and agents for the adjustment of tonicity such as sodium chloride or dextrose.

Pharmaceutical formulations for PRG4 preferably are sterile. Sterilization can be accomplished, for example, by filtration through sterile filtration membranes. Where the composition is lyophilized, filter sterilization can be conducted prior to or following lyophilization and reconstitution. Aqueous solutions may be packaged for use as-is, or lyophilized, the lyophilized preparation being combined with a sterile aqueous carrier prior to administration. The pH of the preparations typically is between 3 and 11, more preferably between 5 and 9 or between 6 and 8, and most preferably between 7 and 8, such as 7 to 7.5. Formulated PRG4 for administration may be packaged in multiple single dose units, each containing a fixed amount of the above-mentioned agent or agents, such as in a sealed package of tablets or capsules.

Pharmaceutical compositions containing PRG4, such as those disclosed herein, can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, and rectal administration. The pharmaceutical compositions are intended for parenteral, intranasal, topical, oral, or local administration, such as by a transdermal means, for therapeutic treatment. The pharmaceutical compositions can be administered parenterally (e.g., by intravenous, intramuscular, or subcutaneous injection), or by oral ingestion, or by topical application or intra-articular injection at areas affected by gout such as the knee, ankle, finger joint, or elbow. Additional routes of administration include intravascular, intra-arterial, intratumor, intraperitoneal, intraventricular, intraepidermal, as well as nasal, ophthalmic, intrascleral, intraorbital, rectal, topical, or aerosol inhalation administration.

This disclosure provides compositions for parenteral administration that comprise the above mentioned agents dissolved or suspended in an acceptable carrier, preferably an aqueous carrier, e.g., water, buffered water, saline, PBS, and the like. The compositions may contain pharmaceutically acceptable auxiliary substances as required to approximate physiological conditions, such as pH adjusting and buffering agents, tonicity adjusting agents, wetting agents, detergents and the like. This disclosure also provides compositions for oral delivery, which may contain inert ingredients such as binders or fillers for the formulation of a tablet, a capsule, and the like. Furthermore, this disclosure provides compositions for local administration, which may contain inert ingredients such as solvents or emulsifiers for the formulation of a cream, an ointment, and the like.

A preferred route of administration for PRG4 is by IV infusion. Useful formulations can be prepared by methods well known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl paraben; antioxidants such as ascorbic acid or sodium bisulfite; chelating agents such as EDTA; buffers such as acetates, citrates or phosphates; and agents for the adjustment of tonicity such as sodium chloride or dextrose.

All references and publications included herein are incorporated by reference. The following examples are not intended to be limiting.

### EXAMPLES

### Example 1: Systemic Administration of rhPRG4 Ameliorates Anemia and Osteoarthritis in Sickle Cell Disease Mice

This example shows systemic administration of rhPRG4 can ameliorate anemia and mitigate osteoarthropathy development in SCD mice.

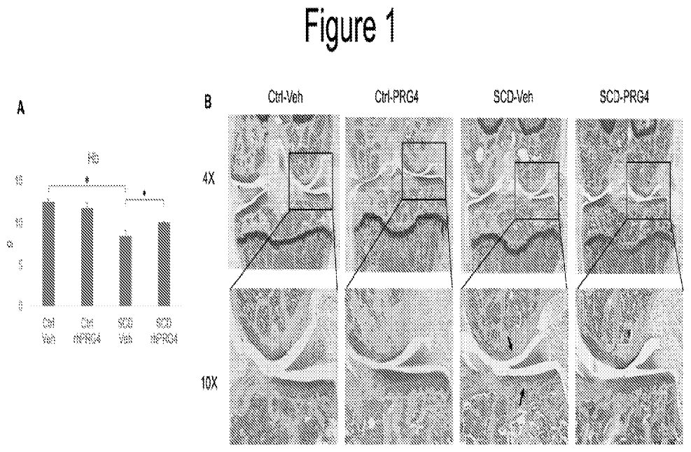

At 6-8 weeks of age control (Ctrl) and SCD male mice were injected subcutaneously with vehicle (PBS with 0.01% Tween20) or rhPRG4 at 0.5 mg/kg BW daily for up to 12 weeks. FIG. 1A shows that four weeks after treatment had commenced, peripheral blood was collected for Hb measurement. Peripheral blood Hb level was significantly decreased in SCD mice compared to Ctrl and was substantially increased in SCD mice with rhPRG4 treatment. n=6-10 mice/group. FIG. 1B shows that six weeks after treatment had commenced, knee samples were collected for histology analysis. Safranin O staining shows loss of proteoglycan in articular cartilage and thinning of articular cartilage of SCD mice compared to Ctrl that was partially rescued with rhPRG4 treatment.

### Example 2: Systemic Administration of rhPRG4 Increases Fetal Hemoglobin (HbF) in Sickle Cell Disease Mice

This example shows systemic administration of rhPRG4 can increase fetal hemoglobin and protects against many of the complications of SCD in mice.

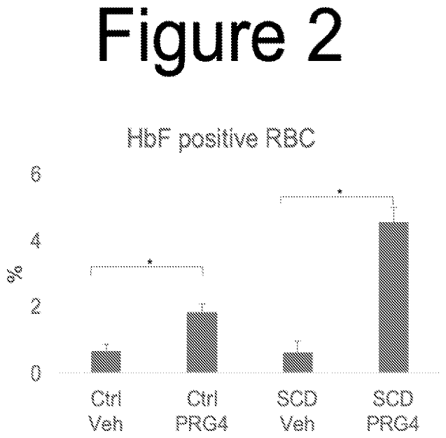

At 6-8 weeks of age control (Ctrl) and SCD male mice were injected subcutaneously with vehicle (PBS with 0.01% Tween20) or rhPRG4 at 0.5 mg/kg BW daily for up to 12 weeks. Twelve weeks after treatment had commenced, peripheral blood was collected. Flow cytometry analysis shows that rhPRG4 treatment significantly increased HbF positive RBC in SCD mice (see, FIG. 2). High levels of fetal hemoglobin protect from many of the complications of SCD and lead to improved survival. n=3-5 mice/group.

### Example 3: Systemic Administration of rhPRG4 Increases Serum and Knee PRG4 in Sickle Cell Disease Mice

This example shows systemic administration of rhPRG4 can increase the amount of serum and knee PRG4 in SCD mice when compared with mice that do not receive rhPRG4 treatment.

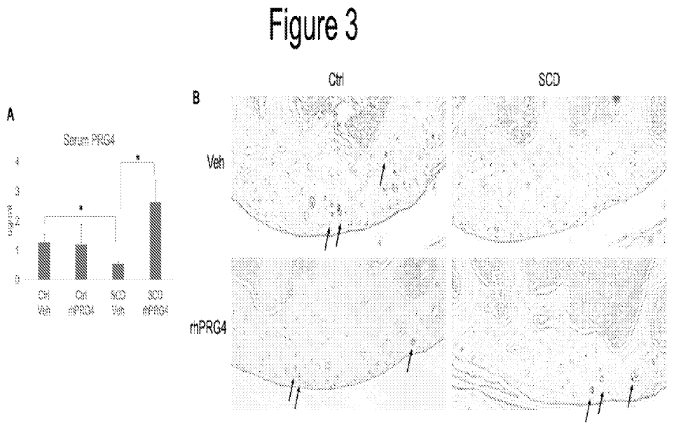

At 6-8 weeks of age control (Ctrl) and SCD male mice were injected subcutaneously with vehicle (PBS with 0.01% Tween20) or rhPRG4 at 0.5 mg/kg BW daily for up to 12 weeks. Six weeks after treatment had commenced, blood was collected. AlphaLISA analysis showed that there is a decrease in serum PRG4 levels in SCD mice compared with Ctrl mice. Sickle cell disease mice treated with rhPRG4 demonstrated increased serum levels of PRG4. N=5 mice/group (See, FIG. 3A). As shown in FIG. 3B, eleven weeks after treatment had commenced, knee samples were harvested for histology. Immunohistochemistry staining shows that there is a decrease in PRG4 expression in knee joint in SCD mice compared with Ctrl mice. Sickle cell disease mice treated with rhPRG4 demonstrated increased PRG4 expression in knee.

### Example 4: Systemic Administration of rhPRG4 Decreases Marrow Area in Sickle Cell Disease Mice

This example shows that there is an increased in marrow area in SCD mice compared to control mice. Mice with SCD treated with rhPRG4 demonstrated decreased marrow area.

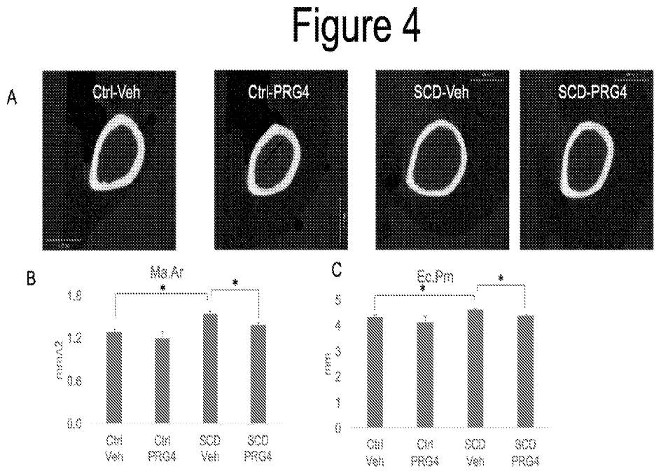

At 6-8 weeks of age control (Ctrl) and SCD male mice were injected subcutaneously with vehicle (PBS with 0.01% Tween20) or rhPRG4 at 0.5 mg/kg BW daily for up to 12 weeks. Eleven weeks after treatment had commenced, bone samples were collected. uCT analysis showed that (see, FIG. 4A-4B) there is increased marrow area (Ma.Ar) and endocortical perimeter (Ec.Pm) in SCD mice compared with Ctrl mice. SCD mice treated with rhPRG4 demonstrated decreased marrow area. n=4/5 mice/group.

### Example 5: Systemic Administration of rhPRG4 Decreases RBC Apoptosis in Sickle Cell Disease Mice

This example shows that there is an increased in apoptotic RBC in SCD mice compared to control mice. Mice with SCD treated with rhPRG4 demonstrated decreased apoptotic RBC.

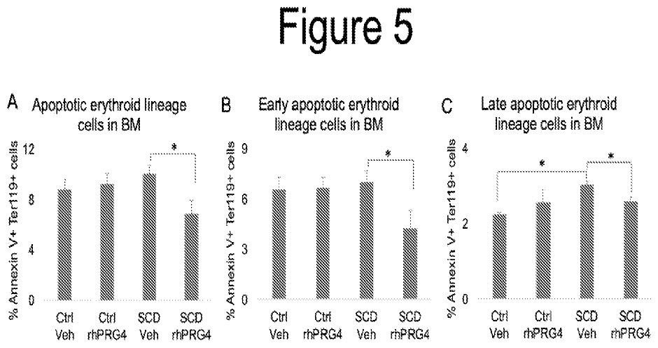

At 6-8 weeks of age control (Ctrl) and SCD male mice were injected subcutaneously with vehicle (PBS with 0.01% Tween20) or rhPRG4 at 0.5 mg/kg BW daily for up to 12 weeks. Six weeks after treatment had commenced, BM was collected and stained with Ter119, Apotracker reagent, and PI for flow cytometry analysis. Flow cytometry shows a significantly higher level of apoptotic erythroid lineage cells in BM of SCD mice compared to Ctrl, that was decreased after rhPRG4 treatment (see, FIG. 5A-5C). n=5 mice/group.

### Example 6: Systemic Administration of rhPRG4 Increased Trabecular Thickness in Sickle Cell Disease Mice

This example shows that there is a decreased trabecular thickness in SCD mice compared to control mice. Mice with SCD treated with rhPRG4 demonstrated increased trabecular thickness.

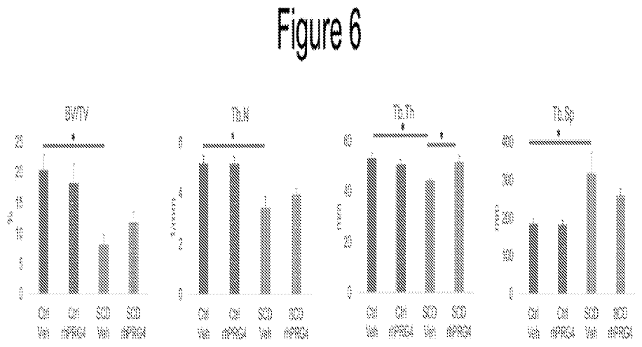

At 6-8 weeks of age control (Ctrl) and SCD male mice were injected subcutaneously with vehicle (PBS with 0.01% Tween20) or rhPRG4 at 0.5 mg/kg BW daily for up to 12 weeks. Eleven weeks after treatment had commenced, bone samples were collected. uCT analysis shows significantly decreased bone volume/total volume (BViTV), trabecular number (Tb.N), trabecular thickness (Tb.Th), and increased trabecular spacing (Tb.Sp) in SCD mice compared with Ctrl. rhPRG4 treatment significantly increased Tb.Th in SCD mice (see, FIG. 6). n=4-5 mice/group.

1. A method of treating sickle cell disease, wherein the method comprises the step of administering an effective amount of PRG4 to a subject in need thereof.

2. The method of claim 2, wherein PRG4 is native or recombinant PRG4.

3. The method of claim 2, wherein the native or recombinant PRG4 is a full-length native PRG4, a recombinant PRG4 or a fragment of the native or recombinant PRG4.

4. The method of claim 1, wherein PRG4 is administered to the subject as a pharmaceutically acceptable composition.

5. The method of claim 1, wherein the subject is suffering from chronic sickle cell disease.

6. The method of claim 1, wherein the subject an acute sickle cell disease.

7. The method of claim 4, wherein the pharmaceutically acceptable composition comprises a pharmaceutically acceptable carrier or diluent.

8. A method of treating anemia, wherein the method comprises the step of administering an effective amount of PRG4 to a subject in need thereof.

9. The method of claim 8, wherein PRG4 is native or recombinant PRG4.

10. The method of claim 9, wherein the native or recombinant PRG4 is a full-length native PRG4, a recombinant PRG4 or a fragment of the native or recombinant PRG4.

11. The method of claim 8, wherein PRG4 is administered to the subject as a pharmaceutically acceptable composition.

12. The method one of claim 8, wherein the anemia is caused by sickle cell disease.

13. The method of claim 12, wherein the anemia is hemolytic anemia.

14. The method of claim 8, wherein the anemia is not caused by sickle cell disease.

15. The method of claim 14, wherein the anemia is iron refractory iron deficiency anemia.

16. The method of claim 11, wherein the pharmaceutically acceptable composition comprises a pharmaceutically acceptable carrier or diluent.
